(19) United States
(12) Patent Application Publication
(10) Pub. No.: US 2015/0366176 A1
(43) Pub. Date: Dec. 24, 2015

(54) Component System Worm Farm

(71) Applicant: Robert Fenchak, Phoenix, AZ (US)

(72) Inventor: Robert Fenchak, Phoenix, AZ (US)

(21) Appl. No.: 14/309,032

(22) Filed: Jun. 19, 2014

(51) Int. Cl. A01K 67/033; C05F 17/02; C05F 17/00

(52) U.S. Cl. CPC A01K 67/033; C05F 17/0009; C05F 17/0258; C05F 17/0205

(57) ABSTRACT

This invention is an apparatus for treating organic matter and/or breeding worms. This invention is a product designed to give an individual the ability to create a worm farm to fit the individual's need, desire, and space. The invention has a component system that allows the individual to make the worm farm bigger or smaller as desired. The system also allows the individual the ability to grow a worm farm outside in a hot climate (such as a summer in Phoenix Ariz.) without the worm farm getting too hot or dried out. This invention allows an individual to keep worms alive in the summer heat of a hot and dry climate such as Arizona. This invention creates a worm farm that is more practical for an urban gardener to have in a residential backyard or side yard.

### CROSS-REFERENCE TO RELATED APPLICATIONS

This application claims the priority of Provisional Application No. 61/838,216, with a filing date of Jun. 21, 2013.

### STATEMENT REGARDING FEDERALLY SPONSORED RESEARCH OR DEVELOPMENT

Not Applicable

### FIELD OF INVENTION

The present invention also relates to an apparatus for the breeding and supply of compost worms. This invention also relates to the production of the fertilized soil and soil fertilizer.

### BACKGROUND

There are many benefits to treating organic matter and breeding worms for personal use. To name a few; the ability to have worms for recreational use such as fishing; to use the organic matter (soil) that has been made fertile from the presence of the worms and worm castings; also, the liquid that passes through the habitat (sometimes referred to as “worm tea”), is extremely rich in nutrients and is desirable by many garden enthusiasts.

In order to grow worms in a hot climate, the worm habitat needs to be larger in area so the soil does not dry out too quickly or get too warm. A type of worm farm which is good for worm production and can be successful in a hot climate outdoors contains large, long trenches and massive amounts of soil and ground space. This type of worm farm is too big to be used by a casual, backyard worm farmer, or urban gardener.

In order for an individual to successfully maintain a worm farm in a hot climate, the amount of soil the worms live in, needs to be larger in area than the stackable worm farms currently available. However, the other worm farm designs which have a larger worm habitat, are too big and would not be possible for many people to have such a farm in their backyard. There is a need to have a worm farm with a large enough amount of soil so worms can survive in a hot climate, but still allow the soil to be manageable by an individual without use of large expensive tools or massive amounts of effort.

This invention makes it possible to increase the size of the worm habitat, but still make the habitat manageable. The modular design of the invention makes it possible to create a worm habitat with only about a 4 foot by 4 foot footprint. However, this habitat can be increased in height and width based on the individual's desire and capabilities due to the component design. This invention can increase the size of the worm farm enough to successfully maintain a worm farm in a hot climate, but still be small enough to keep in a backyard or side yard of a casual, urban gardener.

### SUMMARY OF THE INVENTION

The present invention relates to an apparatus to create; a habitat for worm reproduction, production of worm castings, treatment of organic matter, fertilizing soil, general recreation, and other positive uses. It is a way for an urban gardener to utilize the benefits of a worm farm.

This present invention is also a method to create a worm farm with the ability to attain a habitat in which worms can survive, and thrive, in a hot climate (such as a summer in Phoenix, Arizona) without having to create a very large and permanent worm farm. The component system allows the user to manipulate and rotate the soil and organic matter without use of large tools or large amounts of effort.

The current prior art illustrates a few different ways to create a worm farm. Some prior art allows a user to create a habitat by using a stacking container method. Each container is placed directly on top of another container. Because the worms live at the top layers of the soil, the owner will rotate the stackable containers, to keep the worms at the top and the fertilizing material in the lower containers. However, these types of worm habitats are small and either need to be kept indoors or in a climate where the soil will not get too hot or dry out quickly (when the soil dries out too much, or gets too hot, the words will die). Additionally, these smaller worm farm designs are not practical for worm reproduction, or production of larger amounts of usable fertile soil, to be used for a garden.

This “Component System Worm Farm” (hereafter “invention”) is a component system for treating organic matter and/or breeding worms. This invention allows an individual to grow worms in their backyard in hot climates (climates like those found in Arizona, New Mexico, and parts of inland, southern California). This system is not limited to hot climates and can also be used in other climates as well. The component system allows easy and simple installation and maintenance, which is ideal for the casual worm farmer or urban gardener.

With the use of this invention, an individual will be able to create a worm habitat that will flourish in many climates. The component system will allow the habitat to be made a size that will allow the soil to maintain the desired moisture and temperature for the worms, while also allowing the user to create the habitat in a size that fits his/her needs and abilities.

A key feature of this invention is the component system created by the containers. The component system allows easy setup and maintenance. It allows an individual to temporarily take one container out of the system to use the fertilized soil, and/or worms. The component system also allows an individual to expand the system depending on the individual's desire and space. Having each container as a separate piece to whole apparatus allows the user to make it as small as 2 containers wide, 2 containers long, and 2 containers high. However, the user can expand that system adding more containers reaching the users desired size.

Each container can have the ability to ‘nest’ or temporarily lock into place adjacent to, or on top of, another container. This would prevent any sliding or accidental movement of the container, while still allowing the container to be easily removed from the system and thereafter replaced.

One important factor about any worm farm is that the worms need to be able to freely move about the whole habitat. The worms use all of the soil to move around, breed, and otherwise live in. As the worms do this, the soil becomes more fertile and is ideal for use in gardens and to help other vegetation thrive.

The perforations in each of the containers and on the bottom side of the containers will align with the perforations in the container adjacent. The alignment of the perforations allows worms to freely travel from one container to another. The component system allows the worms to live in the center area of system where the soil would stay moist and cooler, but still allow movement through all the soil within the system.

Another embodiment of the system is the platform on which the containers are placed will have grooves or channels on the top side. The channels will direct the fluid that passes through the system to a specified side of the platform. The channels can also direct the fluid to multiple sides of the platform. Another embodiment of the invention is placing the platform as a slight incline to direct the fluid to one side of the platform. A container can be placed next to the end of the channels (or on the low side of the platform) to collect the fluid and be used at a different location as desired by the user.

The system can have an automated watering and feeding system attached. This watering system can be similar to a drip system or other irrigation system. However, some users might opt to manually water and add organic material as desired.

### BRIEF DESCRIPTION OF THE DRAWINGS

FIG. 1 shows a perspective view of the system.

FIG. 2 shows a side view of the system.

FIG. 3 shows a top view of the system.

FIG. 4 shows a close view of one container used in the system.

### DETAILED DESCRIPTION OF THE DRAWINGS

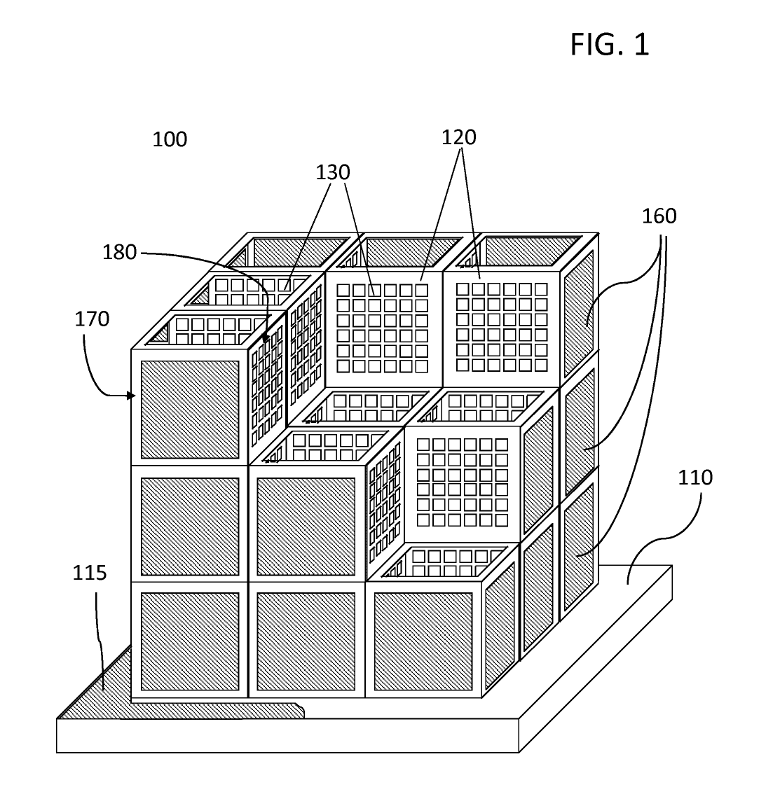

FIG. 1 shows a perspective view of the worm farm system 100. The system 100 has a platform 110 located at the bottom. The platform 110 can comprise of an impermeable material. Another embodiment will have an impermeable cover 115 placed on top of the platform 110. A plurality of containers 120 are placed on top of the platform 110, or if an impermeable cover 115 is used, the containers 120 are placed on top of the impermeable cover 115. The containers 120 are placed to create a substantial cube shape. The containers 120 have perforations 130 on all sides of the container.

The containers 120 can have the ability to stack on top of each other and one enablement allows the containers 120 to partially nest and ‘lock’ into place, so the containers 120 can stack on top of each other without easily falling off. The containers 120 also can be placed side by side. When the containers 120 are placed side by side, the containers will have an outside face 170 and an inside face 180. The perforations 130 on the container's 120 inside face 180 will align with the perforations 130 on the container 120 adjacent, and allow worms to freely pass from one container 120 to another container 120 next to the first container 120. The perforations 130 on each container's 120 bottom side will allow worms to freely pass from one container 120 to another container 120 above and/or below it.

The outside face 170 of the containers 120 will have an impermeable membrane 160. The impermeable membrane 160 will keep the worms, organic material, and any moisture that might present in the system 100, within the system. The moisture will pass down through the organic material to the platform 110.

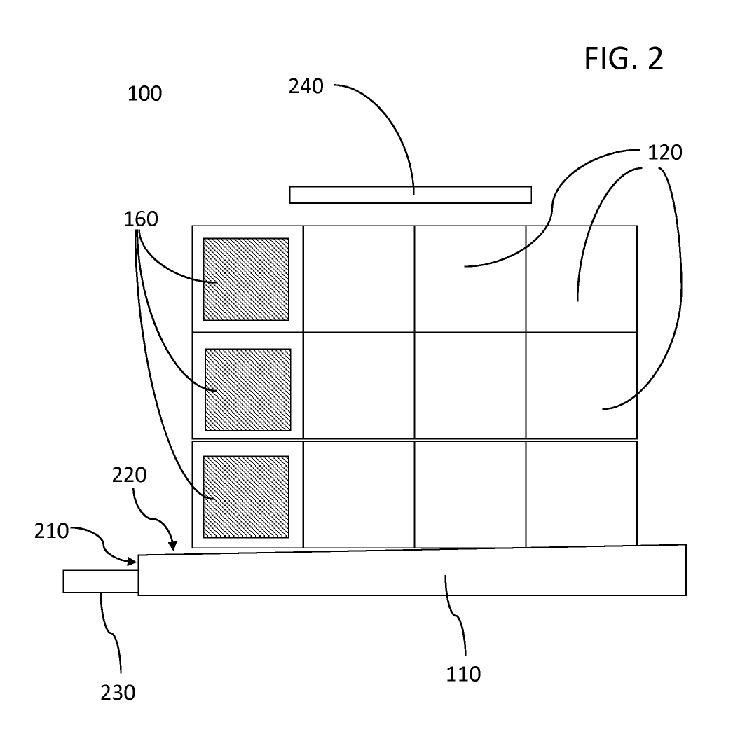

FIG. 2 shows a side view of the system 100. For illustrative purposes, the containers 120 in FIG. 2 are stacked in a way so that the containers 120 are three high and four wide. A feature of this system is that the number of containers 120 can be increased or decreased based on the users space and ability. The containers 120 can be stacked higher (on top of each other) or wider (placed side by side). FIG. 2 illustrates the platform 110 with a sloped top surface 220. This top surface 220 will direct the flow of fluid, which can pass through the system, to a containment area 230 located on one side of the platform 110.

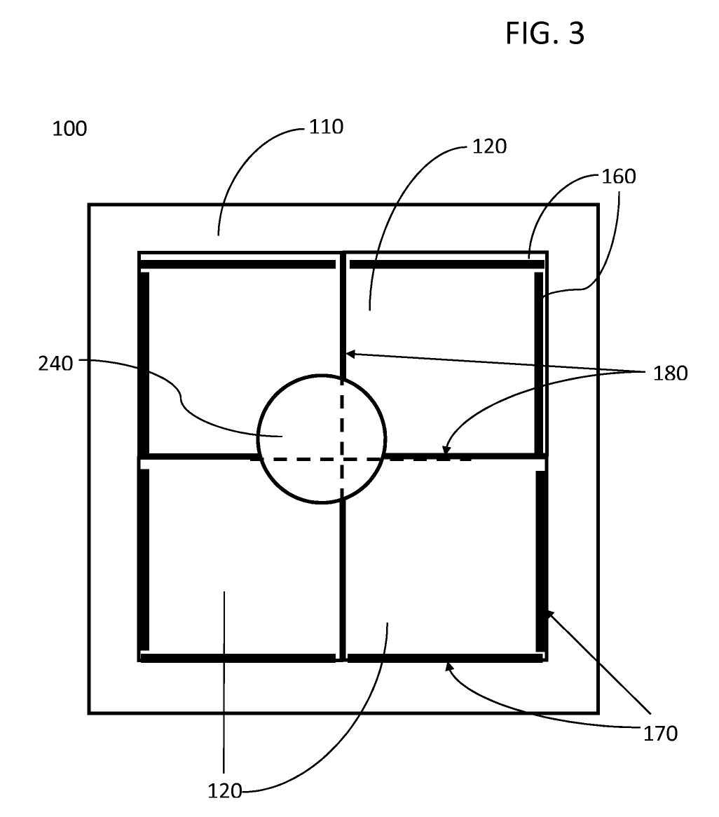

FIG. 3 is a top view of the system 100. An embodiment of the system 100 can have an external watering system 240. The watering system will slowly add moisture to the system 100. FIG. 3 again illustrates that each container 120 will have an inside face 180 and an outside face 170 depending on the arrangement of each container 120 within the system 100.

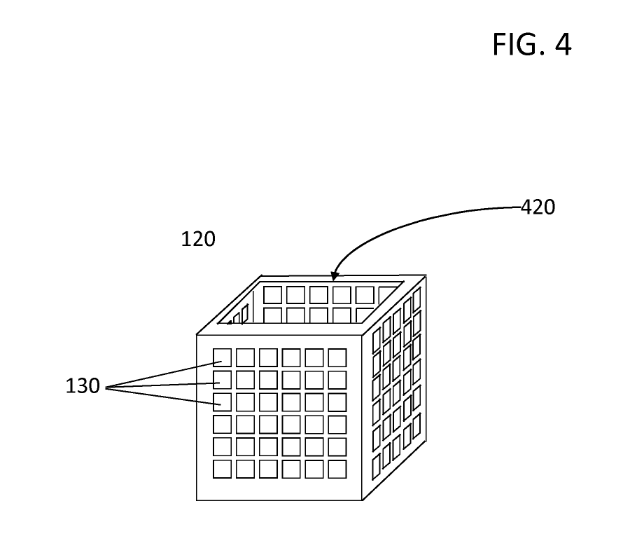

FIG. 4 is a view of one container 120 within the system 100. FIG. 3 illustrates that each of the four sides and the bottom side of the container 120 will have perforations 130. The perforations 130 are large enough to allow worms to freely move from one container to another. The top side 420 of each container 120 will can be completely open for the ease of adding and removing soil and worms.

What claimed is:

1. An apparatus for treating organic matter and/or breeding worms comprising:
a. a platform with an impermeable top surface;
b. a plurality of at least 8 quadrilaterally-faced hexahedron containers, each container having an open top side, a plurality of perforations on each side, and a plurality of perforations on the bottom side, wherein the containers are placed on the top surface of the platform, each container is placed side by side, and/or placed on top of each other forming a substantial hexahedron shape, the placement of each container creates an inside face(s) on each container and outside face(s) on each container (the interior face of the containers are the sides of the container that are adjacent to another container and the exterior face of the containers are the sides of the container that is not adjacent to another container);
c. an impermeable membrane, wherein the membrane is placed on each outside face of each container.

2. The apparatus of claim 1, wherein the perforations are large enough to allow worms to freely pass through the perforations, but prevent passage of substantial quantities of organic matter.

3. The apparatus of claim 1, wherein the containers are substantially the same size and shape.

4. The apparatus of claim 1 further comprising: an external watering system, wherein the external watering system is placed above the top layer of the containers.

5. The apparatus of claim 1, wherein the platform is made of an impermeable material.

6. The apparatus of claim 1, wherein the platform base is covered with an impermeable membrane.

7. The apparatus of claim 1, wherein the platform impermeable top surface is sloped from the horizontal parallel to create a low side of the platform.

8. The apparatus of claim 7, wherein the platform has channels to direct fluid flow to a specified area.

9. The apparatus of claim 7, further comprising: a containment area wherein the containment area is located adjacent the low side of the platform.

10. The apparatus of claim 9 wherein the containment area is comprised of a container which can be removed and replaced.

11. A method of creating a worm farm comprising: placing a platform substantially horizontal; placing a plurality of at least 8 Quadrilaterally-faced hexahedron containers on the platform, placing each container adjacent to another container on the horizontal (‘x’ and ‘z’) axis, creating exterior sides and interior sides of each container (the interior sides of the container are the sides of the container that are adjacent to another container, and the exterior sides of the container are the sides of the container that is not adjacent to another container); placing an impermeable membrane on the exterior side(s) of each container; placing organic material inside the containers; stacking a second layer of containers on top of the existing containers; placing an impermeable membrane on the exterior side(s) of each container of the second layer of containers; placing organic material inside the second layer of containers; adding more layers (3, 4, 5 ect . . .) of containers, adding membranes on the external sides of the containers on each layer, and placing organic material inside each container, on each layer, until desired height is reached.

12. The method of claim 11, wherein the height of the containers does not exceed 6 feet.

13. The method of claim 11 further incorporating an external watering system, wherein the watering system is placed on top of the topmost layer of containers.

14. The method of claim 11 where in the platform is made of impermeable material.

15. The method of claim 11 further comprising, placing an impermeable membrane on top of the platform.

16. The method of claim 11 further comprising: placing a collection device adjacent to the platform.
